(19) United States
(12) Patent Application Publication
(10) Pub. No.: US 2003/0040783 A1
(43) Pub. Date: Feb. 27, 2003

(54) Warming apparatus

(76) Inventor: Andrew Paul Maxwell Salmon, Auckland (NZ)

Correspondence Address: TREXLER, BUSHNELL, GIANGIORGI,, BLACKSTONE & MARR, LTD., 105 W. ADAMS ST., CHICAGO, IL, 60603, US

(21) Appl. No.: 10/242,910

(22) Filed: Sep. 13, 2002

Related U.S. Application Data
(63) Continuation-in-part of application No. 09/662,109, filed on Sep. 14, 2000.

(30) Foreign Application Priority Data
Sep. 21, 1999 (NZ) 337949
Nov. 23, 1999 (NZ) 501245
Sep. 13, 2001 (NZ) 514185

(51) Int. Cl. A61F 7/00

(52) U.S. Cl. 607/111; 607/108

(57) ABSTRACT

An apparatus for raising or maintaining the core temperature of a mammal comprising a radiant heater, for example an LED array for heating the areas of high concentration in Arteriovenous Anastomoses. The LEDs are energised to provide radiant heat energy to the patient hand to achieve a skin temperature with in a predetermined range or a desired core temperature. A pulse oximeter may also be included to sense biological parameters.

This is a Continuation-In-Part of U.S. patent application Ser. No. 09/662,109 filed Sep. 14, 2000.

### BACKGROUND OF THE INVENTION

1. Field of Invention

This invention relates to apparatus and methods of patient warming and in particular to apparatus and methods for maintaining or restoring intra-operative normothermia.

2. Summary of the Prior Art

It is known in the art to provide radiant heating for patients. For example in European Patent Application No. EP 1086673 a method is disclosed for raising or maintaining the core temperature of a mammal during surgery is disclosed. The apparatus comprises a infrared radiant element with a double reflector design to provide a narrow beam of radiation. In His fashion the apparatus is able to direct radiant energy at the mammal's skin in regions with a high concentration of Arteriovenous Anastomoses. The apparatus also includes a skin temperature sensor to allow closed loop control of the heat energy supplied to the mammal.

In Japanese Patent application No. JP 11012819 a warning above is disclosed to relieve the stiffness of the shoulders or pain of hands and give an effect to assist recovery from muscular fatigue or recovery of a joint fiction in playing sports or thereafter by ions generated from electromagnetic waves radiated from far infrared rays from the ceramic, in the glove or to relieve a lesion part by a warming effect, by accelerating bloodstream by the magnetic force or the far infrared rays, in the cold season. However the above systems do not provide an effective, unobtrusive method of heating a patients hand in a suitable fashion to provide core temperature regulation during surgery.

### SUMMARY OF THE INVENTION

It is an object of the present invention to provide an apparatus or method of warming the hand a patient which goes some way to overcoming the abovementioned disadvantages, or which will at least provide the healthcare industry with a useful choice.

Accordingly in a first aspect the present invention consists in apparatus for raising or maintaining the core temperature of a mammal comprising:

a enclosure or biological barrier for a hand (or other high AVA area) of said mammal,

radiant heating means within or adjacent said enclosure adapted to provide radiant heat energy to at least a portion of said hand with high concentration of Arteriovenous Anastomoses to achieve a skin or core temperature within a predetermined range or about a predetermined value.

Preferably said radiant heat energy is radiated with a wavelength in the infrared band.

Preferably said wavelength is between 0.5-2 microns.

Preferably said radiant heating means comprising a plurality of infrared radiant heat sources in use disposed at least about the areas of said hand high in Arteriovenous and Anastomoses.

Preferably said heating elements comprise at least one electrical or electronic light emitting device(s).

Preferably said light emitting device comprises a plurality of energisable LEDs configured to output radiation on energisation of approximately 500 nm wavelength.

Preferably said apparatus further comprising temperature sensing means adapted to sense skin temperature of said hand and said radiant heating means further comprising control means adapted to energise one or more of said LEDs such that said measured skin temperature lies within said predetermined range or about a predetermined value.

Preferably said sensing means further adapted to sense forearm (or other adjacent unheated area of skin) skin temperature and said control means determining a difference value between hand and forearm temperatures, and energising one or more of said LEDs such that said difference value lies within a predetermined range or about a predetermined value.

Preferably when raising the core temperature of said patient said predetermined range of skin temperature is approximately 39° C. to 41° C.

Preferably when maintaining the core temperature of said patient said predetermined range lies about a skin temperature of 37.5° C.

Preferably said apparatus further comprising radiant heat energy reflector means juxtaposed in relation to said radiant heating means such that a substantial portion of the radiant heat energy generated by said radiant heating means is directed at said patient's hand.

Preferably said apparatus further comprises insulation means disposed on or about said radiant heat reflecting means adapted to prevent any substantial transmission of heat energy externally from said apparatus.

Preferably said radiant heating means comprises two tubular elements disposed at a predetermined distance from said hand rest means.

Preferably said tubular elements have a maximum radiant heat energy output of 50 watts each.

Preferably said distance is sufficient to allow the radiant heat energy from said tubular elements to be distributed over said hand.

Preferably said distance is 20 mm.

Preferably said apparatus further comprising a plurality of metal rods adapted to support and generally follow the contour of the underside of said hand.

Preferably said insulation means comprises a thin layer of insulation completely encasing said apparatus.

Preferably said radiant heat reflection means comprises parabolic like reflectors adjacent each said tubular element and adapted to focus the radiant heat energy from said tubular element generally over the area of said patient's hand with a high concentration of Arteriovenous Anastomoses.

Preferably said apparatus is enclosed by an injection moulded thermoplastic case.

Preferably said apparatus further comprising a pulse oximeter juxtaposed adjacent said radiant heating means and adapted to sense at least one parameter(s) relating to said mammal.

Preferably said parameter(s) including pulse rate.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

### BRIEF DESCRIPTION OF THE DRAWINGS

FIG. 1 is a section view of a first embodiment of the present invention in use heating the hand region,

FIG. 2 is a block diagram of the control system according to the present invention,

FIG. 3 is a section view of a second embodiment of the present invention in use heating a hand, and

FIG. 4 is a section view of a third embodiment of the present invention in use heating a hand.

### DETAILED DESCRIPTION OF THE PREFERRED EMBODIMENTS

The present invention is particularly useful for the maintenance of intra-operative normothermia and treatment of hypothermia in surgical patients. Due to space limitations in the surgical area, the traditional heating area of the thoracic region is inaccessible in some circumstances.

The present invention overcomes this by heating the patient in the hand area using infra red heating techniques.

It has been found that, despite this small area, the use of the present invention is effective in not only maintaining normothermia, but also in restoring normothermia in those patients who had become hypothermic, by achieving a net heat gain in the patient. In some patients, due to the type of surgical procedure i.e. beating heart or hip pin apparatus or logistical constraints, it might be very difficult or impossible to effectively treat them via conventional means, i.e. the use of a convective air warmer.

The present invention exploits the properties of one of several specialized areas which the body utilizes for thermoregulation. These are the face/neck, ears, hands and feet. A few millimetres below the surface of the skin in these areas lies the Arteriovenous plexus. The Arteriovenous plexus is a layer of blood vessels which contain Arteriovenous Anastomoses, (AVA), which, when dilated, shunt blood directly from the arterial to the venous system bypassing the capillary beds. This allows the body to shunt a great deal of blood in order to lose heat if the core temperature rises. However, it appears to also allow free access of heat directly into the core circulation if external heat is being applied.

The present invention exploits this portal by raising the temperature of the sub cutaneous vasculature in the hand quickly thereby causing a dilation of the AVA allowing heat energy to be transferred directly into the core circulation The controller allows the unit to maintain a high energy intensity whilst controlling the patient's skin at a safe level.

In humans, AVAs are present in the skin of the hands, feet, ears and nose. In the hands, AVAs are located in the bed of the nails, at the fingertips, on the palm of the phalanxes, and in the nar and hypothenar. The synchronous closing of the AVAs is most likely caused by bursts of efferent sympathetic impulses. The burst frequency is linked to the general heat balance of the body. In situations where there is a need for heat conservation or heat elimination, the AVAs remain mainly closed or mainly open, respectively, resulting in almost nonfluctuation low or high blood velocity values in the afferent arteries. In a thermoneutral situation the AVAs constrict two or three times per minute, causing large, rapid blood velocity fluctuations in the afferent arteries. Vasomotion is believed to be synchronous in all skin AVAS, because blood flow variations in arteries supplying separate areas of skin, such as a hand and a foot, are found to be closely correlated. Fluctuations in blood flow through AVAs also show a close connection with heart rate and arterial blood flow are negatively correlated with fluctuation in mean arterial pressure (MAP) but the fluctuations in MAP precede the blood flow fluctuations by 2-3s.

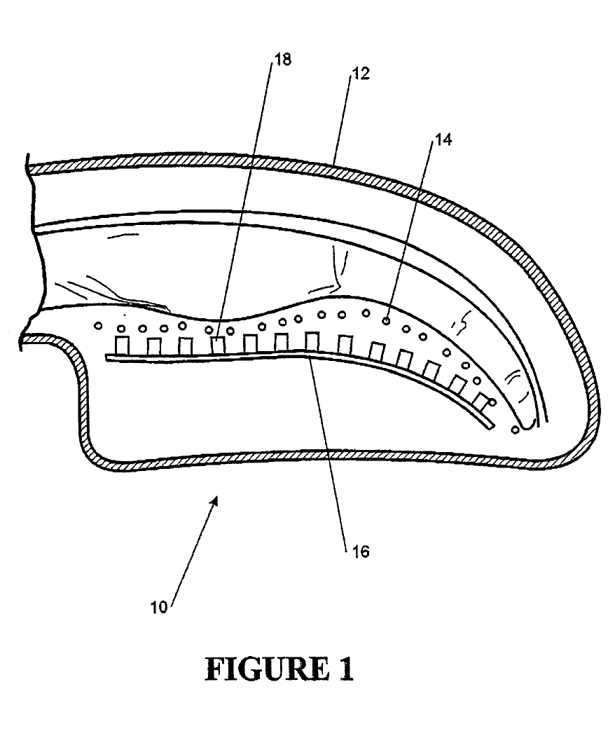

In a first embodiment of the present invention seen in FIG. 1 the apparatus is illustrated heating the patient hand The unit 10 is shown including a plastic or aluminium case 12 which encloses both the hand and the control apparatus. Support structure 14 provides a comfortable contoured rest for the patients hand while allowing substantial radiant heat transmission to either the underside or the topside of the patient's hand. In the preferred embodiment the support structure 14 is provided a plastic material such a silicone or kraton which lies between the heating elements. This provides an environmental seal between the hand and device.

Disposed immediately below support structure 14 is a PCB 16 with a plurality of LEDs 18 mounted thereon LEDs may for example be surface mount or other configurations as is known in the art. PCB 16 is preferably contoured similarly to the support structure 14 but may also be provided flat. Similarity LEDs 18 maybe provided with a concentration in particular areas and/or maybe controlled for different intensities depending on there location and desired effect. Preferably green LEDs are used with a wavelength of approximately 510 nm as experiments have indicated that this is best absorbed by haemoglobin in the blood.

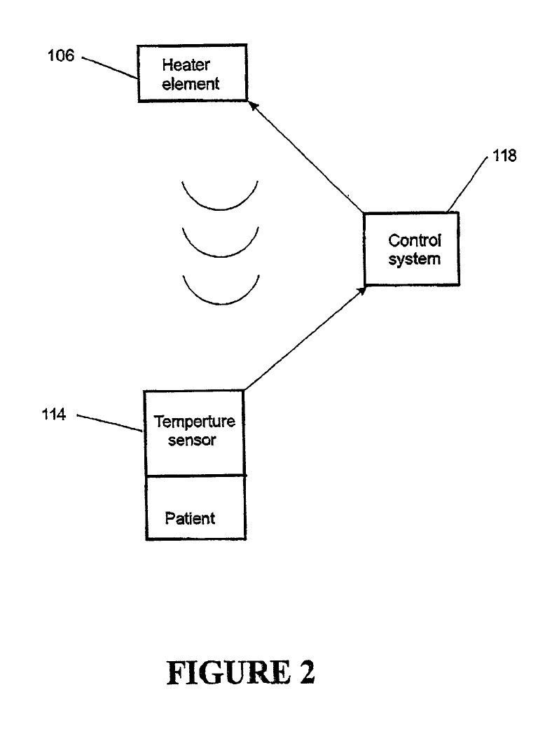

Preferably the set of LEDs has a maximum heat energy output of 100 watts. The control system shown in FIG. 2 provides close loop control over the bank of LEDs. Close loop control either uses skin temperature e.g. as provided by a temperature sensor 114 attached to the underside of the hand or integrated with the support structure 14 or an estimate of core temperature, which maybe determined by detecting the difference between finger temperature and forearm temperature. Preferably the later is to achieve a core temperature of between 36.5 and 37° C.

In a further alternative temperature sensors are provided in a network across the support structure 14 such that the spectrum heating over the entire hand could be detected. The upper limit of energy input would be defined by the hottest region of the hand which could either result in the overall lowering of radiant heating or alternatively could entail a localised reduction in the energisation of LEDs adjacent to that area. In a further alternative an array of infra red sensors on the PCB 16 could be provided to sense the skin temperature of the underside of the hand. In this fashion the LEDs could be instantously switched off and the skin temperature sensed since there will be no residual radiant heat output from the LEDs after switch off.

In order to minimise sterilisation costs a radiant energy transparent bag or other biological barrier which would enclose the hand. The hand may then be able to rest directly on the bank of LEDs 18. Polythene of a suitable thickness is one example of an appropriate material.

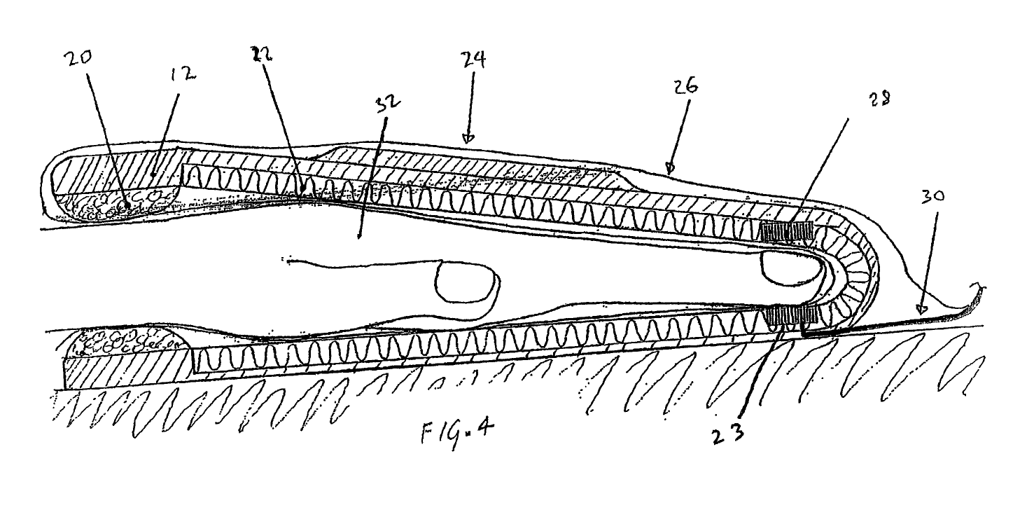

A further application of detecting vital signs of the patient is possible using pulse oximetry. Pulse oximetry is a simple non-invasive method of monitoring the percentage of haemoglobin which is saturated with oxygen as well as pulse beat, calculated heart rate and optionally blood flow through that area, among other things. In FIG. 4 the sensor emitter pad 28 located at the end of one finger, is mounted with LEDs of two different wavelengths for example a further LED in the range of 800 nm as well as sensors to detect the absorption at each wavelength. The light will be partially absorbed by the haemoglobin depending on whether it is saturated or desaturated with oxygen. Also seen in FIG. 4 are wrist pads 20 to keep the hand 32 stationary. The radiant heating array 22 (e.g. LEDs) surrounds the hand and is enclosed by case 12. The disposable cover 26 provides a biological barrier between the hand 32 and the device 10 and covering the exterior of case 12. Display control module 24 on the exterior allows the user or clinician to set temperature, configuration, area and/or period of heating.

By calculating the absorption at the sensor detector pad 23 (opposite side of finger) of each wavelength the proportion of haemoglobin which is oxygenated can be calculated this reading in turn will vary with a pulse rate flow and from this pulse beat and flow can be determined. Data cable 30 connects the oximeter to an analysis module for example a heart rate monitor.

In a further alternative a plasma or TFT type element could be provided to control heating and be more exact or smaller areas. Also the intensity and/or wavelength of the radiant energy could be controlled in very specific zones.

In this fashion when the skin temperature is below the desired range, the heater element 106 is supplied with a voltage known to give the ideal wavelength of radiant energy at the controller 118. Once the skin temperature reaches the desired range the heater element 106 is switched off by the controller 118.

The present invention may be employed in one of two modes. Firstly, it may be used to initially raise the core temperature of a patient. In this case the skin temperature might be set using the interface with the controller 118 to range between 39° C. and 41° C. In this mode there will be a positive net energy transfer between the environment and the patient resulting in patient's core temperature rising. Once the patient's core temperature has reached an acceptable level the present invention may be employed in a second mode whereby it is used to maintain the core temperature of the patient. In this case the skin temperature might be set using the interface with the controller 118, for example at 37.5° C. which would result in a roughly zero net energy transfer between the patient and the environment. In this case the radiant warmer is only compensating for the heat losses of the patient.

It will be appreciated that in the normal course of surgery the initial skin temperature might be set quite high and then as the core temperature of the patient rises to that approaching the set skin temperature would be slowly titrated down to a maintenance level. In a further embodiment a core temperature probe maybe used to feed back to the controller actual core temperature so that direct compensation for core temperature can be made.

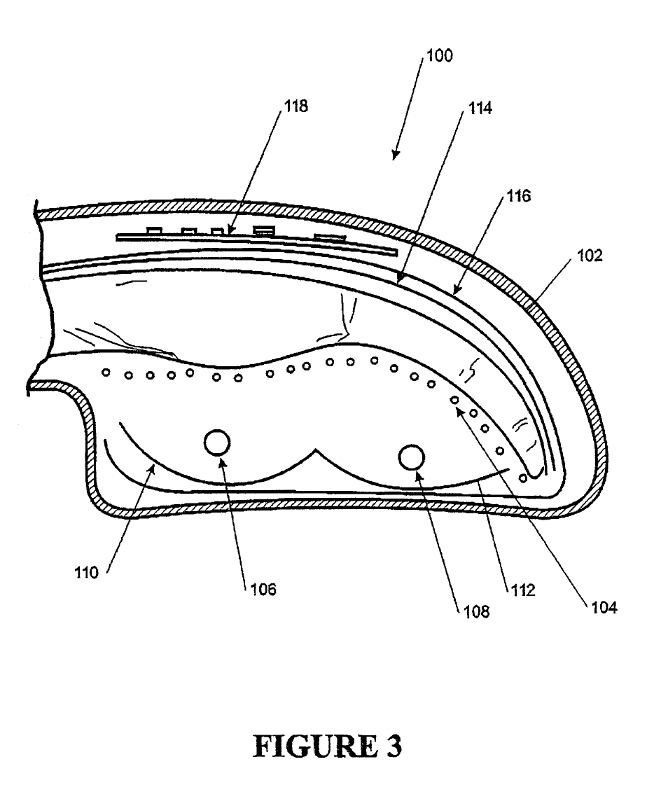

In a second embodiment of the present invention seen in FIG. 3 the apparatus is illustrated heating the patient's hand. The unit 100 is shown including a injection moulded thermoplastic or aluminum case 102. The case 102 is robust and of a size designed to accommodate most size hands of humans. Inside the case 102 are a number of stainless steel support rods 104 with a contoured profile to allow the patient to comfortably rest their hand on top of them. The support rods 104 provide a cool surface for the hand to rest on while still allowing substantial radiant heat transmission to the underside of the patient's hand.

Approximately 20 mm underneath said support rods are two tubular elements 106, 108 running laterally across the width of the unit. Each of the tubular elements is rated to a maximum output of 50 watts providing infrared radiation at a wavelength of approximately 2 microns. Underneath each tubular element is a polished aluminium infrared radiation reflector 110, 112. Each reflector 110, 112 is constructed from polished aluminium which has a high reflectivity but low emissivity for infrared radiation of this wavelength. The shape of the reflector resembles a parabolic shape but is specifically calculated to spread the radiation evenly over the areas of the hand having a high concentration of Arteriovenous Anastomoses. The exact shape (not strictly parabolic) can be easily calculated using any one of a number of commercially available mathematical simulation packagings, run on a computer or any other method as is known in the art. A further reflector 114 is provided directly above the hand to reflect any stray radiation which passes through the fingers or around the hand back at the hand.

While the reflectors are designed to minimise any loss of radiation heat energy not transferred to the hand, there will always be some losses. Accordingly in the preferred embodiment a layer of insulation 116 is provided around the reflectors generally enclosing the apparatus inside the case 102. Preferably the insulation is NOMEX™ brand insulation disposed on the inner surface of the case 102.

Referring to FIG. 2 the system control can also be used for controlling the heater element 106 of the second embodiment. The heater element 106 is a solid cylindrical member which is typically heated to a maximum temperature of 1200° C. which results in the IR radiation of 2 micron wavelength however anything in the range of 0.5-2 micros is desirable. The heater element 106 is electrically connected to the warmer controller 118 which utilises closed loop control of the input desired skin temperature as compared against the measured skin temperature, using the temperature sensor 114.

It will also be appreciated that such a method is not limited specifically to human and is generally applicable for use with animals generally, although particularly with warm blooded mammals.

We believe that this method of heating is unique to the radiant warmer and cannot be achieved by the current warming technique of choice which is convective air warming. This is due to several reasons:

1. To achieve the necessary vasodilation and consequent dilation of the AVA, a high energy source is needed to raise the sub-dermal temperature significantly.

2. To maintain this sub-dermal temperature necessary for vasodilation, without risk of overheating causing injury, some form of patient feedback mechanism is required together with accurate and effective response. The present invention uses a controller and skin temperature sensor to monitor skin temperature and adjust the unit's heat output. The radiant nature of the heat from the warmer gives near instantaneous control of heat output to the patient. This allows the Radiant system to achieve and maintain the desired skin temperature safely. Convective air warmers do not have a patient feedback system and so have no way of controlling skin temperature. By default, therefore, they achieve a lower skin temperature as, without control, they run a risk of causing thermal injury to the patient.

3. In the preferred embodiment of the present invention the patient is radiated with IR at a peak wavelength of 0.5-2 microns. This wavelength achieves a penetration through the skin of approximately 1-10 mm, allowing energy to be transferred directly into the tissue. This raises the temperature of the tissue quickly, rapidly establishing the desired vasodilation of the AVA, and allows transfer of heat energy directly into the circulation. Convective air warmers however, pass their energy through the skin's surface via conduction slowing the transfer of the energy into the deeper tissues and the circulatory system, and limiting the safe transfer rate.

Thus it will be appreciated that what is described is an effective method and apparatus of heating a patient during surgery. In the preferred embodiment the hand area is heated, however other areas which have high AVA concentration, may also be used. The apparatus allows excellent regulation of the patient's core temperature throughout surgery and is unobtrusive allowing good access for the surgical team.

1. An apparatus for raising or maintaining the core temperature of a mammal comprising:
a enclosure or biological barrier for a hand (or other high AVA area) of said mammal,
radiant heating means within or adjacent said enclosure adapted to provide radiant heat energy to at least a portion of said hand with high concentration of Arteriovenous Anastomoses to achieve a skin or core temperature within a predetermined range or about a predetermined value.

2. An apparatus as claimed in claim 1 wherein said radiant heat energy is radiated with a wavelength in the infrared band.

3. An apparatus as claimed in claim 2 wherein said wavelength is between 0.5-2 microns.

4. An apparatus as claimed in claim 3 wherein said radiant heating means comprising a plurality of infrared radiant heat sources in use disposed at least about the areas of said hand high in Arteriovenous and Anastomoses.

5. An apparatus as claimed in claim 4 wherein said heating elements comprise at least one electrical or electronic light emitting device(s).

6. An apparatus as claimed in claim 5 wherein said light emitting device comprises a plurality of energisable LEDs configured to output radiation on energisation of approximately 500 nm wavelength.

7. An apparatus as claimed in claim 6 further comprising temperature sensing means adapted to sense skin temperature of said hand and said radiant heating means further comprising control means adapted to energise one or more of said LEDs such that said measured skin temperature lies within said predetermined range or about a predetermined value.

8. An apparatus as claimed in claim 7 wherein said sensing means further adapted to sense forearm (or other adjacent unheated area of skin) skin temperature and said control means determining a difference value between hand and forearm temperatures, and energising one or more of said LEDs such that said difference value lies within a predetermined range or about a predetermined value.

9. An apparatus as claimed in claims 7 or 8 wherein when raising the core temperature of said patient said predetermined range of skin temperature is approximately 39° C. to 41° C.

10. An apparatus as claimed in claim 9 wherein when maintaining the core temperature of said patient said predetermined range lies about a skin temperature of 37.5°0 C.

11. An apparatus as claimed in claim 1 further comprising radiant heat energy reflector means juxtaposed in relation to said radiant heating means such that a substantial portion of the radiant heat energy generated by said radiant heating means is directed at said patient's hand.

12. An apparatus as claimed in claim 11 further comprises insulation means disposed on or about said radiant heat reflecting means adapted to prevent any substantial transmission of heat energy externally from said apparatus.

13. An apparatus as claimed in claim 12 wherein said radiant heating means comprises two tubular elements disposed at a predetermined distance from said hand rest means.

14. An apparatus as claimed in claim 13 wherein said tubular elements have a maximum radiant heat energy output of 50 watts each.

15. An apparatus as claimed in claim 14 wherein said distance is sufficient to allow the radiant heat energy from said tubular elements to be distributed over said hand.

16. An apparatus as claimed in claim 15 wherein said distance is 20 mm.

17. An apparatus as claimed in claim 16 further comprising a plurality of metal rods adapted to support and generally follow the contour of the underside of said hand.

18. An apparatus as claimed in claim 12 wherein said insulation means comprises a thin layer of insulation completely encasing said apparatus.

19. An apparatus as claimed in claim 11 wherein said radiant heat reflection means comprises parabolic like reflectors adjacent each said tubular element and adapted to focus the radiant heat energy from said tubular element generally over the area of said patient's hand with a high concentration of Arteriovenous Anastomoses.

20. An apparatus as claimed in any one of claims 1 to 17 wherein said apparatus is enclosed by an injection moulded thermoplastic case.

21. An apparatus as claimed in any one of claims 1 to 17 further comprising a pulse oximeter juxtaposed adjacent said radiant heating means and adapted to sense at least one parameter(s) relating to said mammal.

22. An apparatus as claimed in claim 21 wherein said parameter(s) including pulse rate.
